(19) United States
(12) Patent Application Publication
(10) Pub. No.: US 2014/0256044 A1
(43) Pub. Date: Sep. 11, 2014

(54) CULTURE MEDIA

(71) Applicant: MAYO FOUNDATION FOR MEDICAL EDUCATION AND RESEARCH, Rochester, MN (US)

(72) Inventor: Laura Marlow, Jacksonville, FL (US)

(21) Appl. No.: 14/190,733

(22) Filed: Feb. 26, 2014

Related U.S. Application Data
(60) Provisional application No. 61/774,402, filed on Mar. 7, 2013.

(51) Int. Cl. C12N 5/09

(52) U.S. Cl. CPC C12N 5/0693
USPC 435/406

(57) ABSTRACT

This document provides methods and materials involved in creating immortalized cell lines (e.g., immortalized breast, ovarian, or prostate cancer cell lines) and culturing immortalized cell lines. For example, culture media that can be used to create immortalized breast, ovarian, or prostate cancer cell lines are provided.

### CROSS-REFERENCE TO RELATED APPLICATIONS

This application claims the benefit of U.S. provisional application 61/774,402, filed Mar. 7, 2013. The disclosure of the prior application is considered part of (and is incorporated by reference in) the disclosure of this application.

### BACKGROUND

1. Technical Field

This document relates to methods and materials involved in creating immortalized cell lines (e.g., immortalized breast, ovarian, or prostate cancer cell lines) and culturing immortalized cell lines. For example, this document provides culture media that can be used to create immortalized breast, ovarian, or prostate cancer cell lines.

2. Background Information

Creating immortalized cell lines can be very tedious and can have a high failure rate, thereby resulting in a limited number of particular cancer cell lines. In some cases, available cell lines can be very slow growing, can fail to grow to confluence, and can shed into the media.

### SUMMARY

This document provides methods and materials involved in creating immortalized cell lines (e.g., immortalized breast, ovarian, or prostate cancer cell lines) and culturing immortalized cell lines. For example, this document provides culture media that can be used to create immortalized breast, ovarian, or prostate cancer cell lines. As described herein, culture media containing the ingredients provided herein can be used to create immortalized breast, ovarian, or prostate cancer cell lines.

In general, one aspect of this document features a culture medium preparation for creating an immortalized breast, ovarian, or prostate cell line. The preparation comprises, or consists essentially of, (a) at least three (e.g., at least three, four, five, six, seven, eight, nine, ten, eleven, or twelve) compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine, and (b) at least three (e.g., at least three, four, five, six, seven, eight, nine, ten, or eleven) compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

In another aspect, this document features a method for creating an immortalized breast, ovarian, or prostate cell line. The method comprises, or consists essentially of, culturing breast, ovarian, or prostate primary cells in the presence of a culture medium preparation. The preparation comprises, or consists essentially of, (a) at least three (e.g., at least three, four, five, six, seven, eight, nine, ten, eleven, or twelve) compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine, and (b) at least three (e.g., at least three, four, five, six, seven, eight, nine, ten, or eleven) compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains photographs of breast, ovarian, and prostate cells cultured with BROV medium or MEGM medium (breast), DMEM medium (ovarian), or PrEGM medium (prostate).

FIGS. 2A-C contain graphs plotting growth rates for breast (A), ovarian (B), and prostate (C) cells cultured with BROV medium or MEGM medium (breast), DMEM medium (ovarian), or PrEGM medium (prostate).

FIG. 3 contains photographs of four different breast cell lines cultured with BROV medium.

FIG. 4 contains photographs of four different ovarian cell lines cultured with BROV medium.

FIG. 5 contains photographs of four different prostate cell lines cultured with BROV medium.

FIG. 6 contains graphs plotting growth rates for breast (MCBJ2 cells) and prostate (PRJ-71T cells) cells cultured with αBROV medium, BROV medium, or DMEM medium.

### DETAILED DESCRIPTION

This document provides methods and materials involved in creating immortalized cell lines (e.g., immortalized breast, ovarian, or prostate cancer cell lines) and culturing immortalized cell lines. For example, this document provides culture media that can be used to create immortalized breast, ovarian, or prostate cancer cell lines. As described herein, culture media containing the ingredients provided herein can be used to create immortalized breast, ovarian, or prostate cancer cell lines with a success rate that is greater than 2 percent (e.g., greater than 2, 3, 4, 5, 6, 7, or 8 percent). For example, the culture media provided herein can be used to create immortalized breast, ovarian, or prostate cancer cell lines with a success rate that is between about 2 percent and about 15 percent (e.g., between about 2 percent and about 12 percent, between about 2 percent and about 10 percent, between about 2 percent and about 8 percent, between about 3 percent and about 15 percent, between about 3 percent and about 12 percent, between about 3 percent and about 10 percent, between about 3 percent and about 8 percent, between about 4 percent and about 15 percent, between about 4 percent and about 12 percent, between about 4 percent and about 10 percent, between about 4 percent and about 8 percent, between about 5 percent and about 8 percent).

A culture medium preparation can include a base medium or a combination of base media. For example, a culture medium preparation provided herein can include a mixture of MEBM, DMEM, and F12 media or a mixture of alpha minimum essential medium (αMEM), DMEM, and F12 media.

In some cases, a culture medium preparation provided herein can include a mixture of MEBM Basal Media without Phenol Red (Part Code CC-3153; Lonza, Verviers, Belgium) and DMEM/F12 (50:50) medium (Catalog No. 16-405-CV; Mediatech, Inc., A Corning Subsidiary, Manassas, Va.). For example, a culture medium preparation provided herein can include a mixture of 500 mL of MEBM Basal Media without Phenol Red (Part Code CC-3153; Lonza, Verviers, Belgium) and 500 mL of DMEM/F12 (50:50) medium (Catalog No. 16-405-CV; Mediatech, Inc., A Corning Subsidiary, Manassas, Va.).

In some cases, a culture medium preparation provided herein can include a mixture of αMEM (Catalog No. SH30265.01; Thermo Scientific, Logan, Utah) and DMEM/F12 (50:50) medium (Catalog No. 16-405-CV; Mediatech, Inc., A Corning Subsidiary, Manassas, Va.). For example, a culture medium preparation provided herein can include a mixture of 500 mL of αMEM (Catalog No. SH30265.01; Thermo Scientific, Logan, Utah) and 500 mL of DMEM/F12 (50:50) medium (Catalog No. 16-405-CV; Mediatech, Inc., A Corning Subsidiary, Manassas, Va.). Such a mixture of base media can have a combination of base media ingredients as listed in Table 1.

TABLE 1
Combination of base media ingredients.

| Ingredients for αMEM | mg/L | mmol/L |
|---|---|---|
| Inorganic Salts | | |
| Calcium Chloride (anhydrous) | 200 | 1.8026 |
| Potassium Chloride | 400 | 5.3655 |
| Magnesium Sulfate (anhydrous) | 97.67 | 0.8112 |
| Sodium Chloride | 6800 | 116.3587 |
| Sodium Phosphate Monobasic | 140 | 1.0146 |
| H₂O | | |
| Amino Acids | | |
| L-Alanine | 25 | 0.2806 |
| L-Arginine-HCl | 126.98 | 0.6028 |
| L-Asparagine-H₂O | 50 | 0.333 |
| L-Aspartic Acid | 30 | 0.2254 |
| L-Cysteine-HCl—H₂O | 100 | 0.5693 |
| L-Cystine-2HCl | 31.28 | 0.0999 |
| L-Glutamic Acid | 75 | 0.5098 |
| L-Glutamine | 292 | 1.9979 |
| Glycine | 50 | 0.666 |
| L-Histidine-HCl—H₂O | 41.88 | 0.1998 |
| L-Isoleucine | 52.5 | 0.4002 |
| L-Leucine | 52.4 | 0.3995 |
| L-Lysine-HCl | 72.47 | 0.3968 |
| L-Methionine | 15 | 0.1005 |
| L-Phenylalanine | 32 | 0.1937 |
| L-Proline | 40 | 0.3474 |
| L-Serine | 25 | 0.2379 |
| L-Threonine | 48 | 0.403 |
| L-Tryptophan | 10 | 0.049 |
| L-Tyrosine-2NA-2H₂O | 51.9 | 0.1987 |
| L-Valine | 46 | 0.3927 |
| Vitamins | | |
| L-Ascorbic Acid | 50 | 0.2839 |
| D-BIOTIN | 0.1 | 0.0004 |
| Calcium D-Pantothenate | 1 | 0.0021 |
| Choline Chloride | 1 | 0.0072 |
| Folic Acid | 1 | 0.0023 |
| Myo-Inositol | 2 | 0.0111 |
| Niacinamide | 1 | 0.0082 |
| Pyridoxal Hydrochloride | 1 | 0.0049 |
| Riboflavin | 0.1 | 0.0003 |
| Thiamine-HCl | 1 | 0.003 |
| Vitamin B12 | 1.36 | 0.001 |
| Other Ingredients | | |
| Adenosine | 10 | 0.0374 |
| Cytidine | 10 | 0.0411 |
| D-Glucose (anhydrous) | 1000 | 5.5506 |
| Guanosine | 10 | 0.0353 |
| DL-Alpha-Lipoic Acid | 0.2 | 0.001 |
| Phenol Red-NA | 11 | 0.0292 |
| Sodium Pyruvate | 110 | 0.9996 |
| Thymidine | 10 | 0.0413 |
| 2′-Deoxyadenosine H₂O | 10 | 0.0371 |
| 2′-Deoxycytidine HCl | 11 | 0.0417 |
| 2′-deoxyguanosine | 10 | 0.0351 |
| Uridine | 10 | 0.041 |
| Sodium Bicarbonate | 2200 | 26.1874 |

| Ingredients for DMEM/F12 | mg/L |
|---|---|
| Inorganic Salts | |
| CaCl₂ (anhydrous) | 116.65 |
| CuSO₄ (anhydrous) | 0.0008 |
| Fe(NO₃)₃•9H₂O | 0.05 |
| KCl | 311.80 |
| MgSO₄ (anhydrous) | 84.95 |
| NaCl | 7000.00 |
| NaH₂PO₄•H₂O | 62.50 |
| Na₂HPO₄ (anhydrous) | 71.00 |
| NaHCO₃ | 2438.00 |
| ZnSO₄•7H₂O | 0.4315 |
| Amino Acids | |
| L-Alanine | 4.45 |
| L-Arginine•HCl | 147.50 |
| L-Asparagine•H₂O | 7.50 |
| L-Aspartic acid | 6.65 |
| L-Cysteine•HCl•H₂O | 17.56 |
| L-Cystine•2HCl | 31.285 |
| L-Glutamic acid | 7.25 |
| L-Glutamine | 365.10 |
| Glycine | 18.75 |
| L-Histidine•HCl•H₂O | 31.48 |
| L-Isoleucine | 54.37 |
| L-Leucine | 58.95 |
| L-Lysine•HCl | 91.35 |
| L-Methionine | 17.24 |
| L-Phenylalanine | 35.48 |
| L-Proline | 17.25 |
| L-Serine | 26.25 |
| L-Threonine | 53.55 |
| L-Tryptophan | 9.02 |
| L-Tyrosine•2Na•2H₂O | 55.815 |
| L-Valine | 52.85 |
| Vitamins | |
| Biotin | 0.00365 |
| D-Calcium pantothenate | 2.24 |
| Choline Chloride | 8.98 |
| Folic Acid | 2.65 |
| i-Inositol | 12.61 |
| Nicotinamide | 2.0185 |
| Pyridoxine•HCl | 2.031 |
| Riboflavin | 0.219 |
| Thiamine•HCl | 2.17 |
| Vitamin B12 | 0.68 |
| Other Ingredients | |
| D-Glucose | 3151.00 |
| Hypoxanthine, Na | 2.385 |
| DL-Thioctic Acid | 0.105 |
| Methyl lineoleate | 0.044 |
| Putrescine•2HCl | 0.08 |
| Sodium pyruvate | 110.00 |
| Thymidine | 0.365 |
| Specifications | |
| pH (after buffer) | 7.2 ± 0.2 |
| Osmolality (mOsm/kg) | 310 ± 30 |

In some cases, a culture medium preparation provided herein can include a base medium having the combination of base media ingredients as listed in Table 1 with the amount of each ingredient being within a range of no more than 8 percent (e.g., no more than 7, 6, 5, 4, 3, 2, or 1 percent) of the amount indicated in Table 1 for each ingredient. For example, a culture medium preparation provided herein can include a base medium having each ingredient listed in Table 1 at either the amount indicated in Table 1 for a particular ingredient or at an amount that is within 5 percent of the amount indicated in Table 1 for a particular ingredient.

A culture medium preparation provided herein can include a combination of supplement ingredients added to the base medium. Examples of such supplement ingredients are provided in Table 2. In some cases, a culture medium preparation provided herein can include all the ingredients listed in Table 2 within the ranges indicated. In some cases, a culture medium preparation provided herein can include all the ingredients listed in Table 2 at the exemplary final concentration indicated.

TABLE 2
Culture medium preparation ingredients
per 1000 mL of base medium.

| | | Exemplary Final | |
|---|---|---|---|
| Ingredient | Range | Concentration | |
| HEPES buffer | | 10 | mM |
| bovine pituitary extract | 10 to 50 μg/mL | 26 | μg/mL |
| human epithelial growth | 0.2 to 10 ng/mL | 1.5 | ng/mL |
| factor | | | |
| hydrocortisone | 0.1 to 1 μg/mL | 0.25 | μg/mL |
| amphotericin B | 0.1 to 0.5 μg/mL | 0.25 | μg/mL |
| insulin | 1 to 5 μg/mL | 2 | μg/mL |
| L-glutamine | 100 to 300 mM | 200 | mM |
| penicillin | 50 to 150 IU | 100 | IU |
| streptomycin | 50 to 150 μg/mL | 100 | μg/mL |
| non-essential amino acids | 0.5 to 2X | 1X | |
| beta-estradial | 0.5 to 10 nM | 1 | nM |

| | | |
|---|---|---|
| fetal bovine serum | 1 to 5% | 2% |
| AlbuMAX ®I | 0.01 to 0.5% | 0.1% |

or the following item in place of AlbuMAX ®I:

| | | | |
|---|---|---|---|
| Lipid-rich bovine serum | 0.005 to 0.05 g | 0.01 | g |
| albumin | | | |

| | | |
|---|---|---|
| B-27 | 5 to 20 mL (0.25 to 1X) | 0.5 X |

or the following items in place of B-27:

| | |
|---|---|
| Biotin | 0.01 to 1 mg/L |
| DL Alpha Tocopherol | 0.01 to 1 mg/L |
| Acetate | |
| DL Alpha-Tocopherol | 0.01 to 1 mg/L |
| Vitamin A (acetate) | 0.01 to 1 mg/L |
| BSA, fatty acid free | 1 to 5 mg/mL |
| Fraction V | |
| Catalase | 0.005 to 0.05 μg/mL |
| Human Recombinant | 1 to 200 mg/L |
| Insulin | |
| Superoxide Dismutase | 0.001 to 1 μM |
| Corticosterone | 0.01 to 0.2 μg/mL |
| D-Galactose | 1 to 1000 mg/L |
| Ethanolamine HCl | 0.01 to 0.05 mM |
| Glutathione (reduced) | 1 to 100 ng/mL |
| L-Carnitine HCl | 1 to 200 μg/mL |
| Linoleic Acid | 0.01 to 1 mg/L |
| Linolenic Acid | 0.01 to 1 mg/L |
| Progesterone | 1 to 100 nM |
| Putrescine 2HCl | 1 to 500 μM |
| Sodium Selenite | 1 to 50 nM |
| T3 (triodo-I-thyronine) | 0.1 to 10 nM |

| | | |
|---|---|---|
| N-2 | 2.5 to 10 mL (0.25 to 1X) | 5 mL (0.5 X) |

or the following items in place of N-2:

| | | | |
|---|---|---|---|
| Human Transferrin | 1 to 200 mg/L | 50 | mg/L |
| (Holo) | | | |
| Insulin Recombinant | 1 to 50 mg/L | 2.5 | mg/L |
| Full Chain | | | |
| Progesterone | 1 to 100 nM | 10 | nM |
| Putrescine | 1 to 500 μM | 50 | μM |
| Selenite | 1 to 50 nM | 15 | nM |

Any appropriate method can be used to produce a culture medium preparation provided herein. For example, standard sterile culture medium mixing techniques can be used to make a culture medium preparation provided herein. Once made, a culture medium preparation provided herein can be used to culture cells (e.g., immortalized cell lines such as immortalized breast, ovarian, or prostate cancer cell lines). In some cases, a culture medium preparation provided herein can be used to create immortalized cell lines (e.g., immortalized breast, ovarian, or prostate cancer cell lines) from primary cancer cells. For example, a portion of a patient tumor tissue can be finely minced, washed in PBS, and cultured in a medium preparation provided herein at, for example, about 37° C. in a humidified atmosphere with about 5% CO₂. After about a week in culture, the tissues can be digested in about 0.25% trypsin (from, e.g., Cellgro) for about 10 minutes at about 37° C. followed by a wash with PBS. The cells can be cultured for about 6 to 12 months and can be designated as being a stable cell line once they reach about passage 20 or when they are homogeneous. In some cases, the cells can be exposed to selection for “tumor initiating cells” (TICs) by plating the cells in plates (e.g., low affinity plates) for promoting suspension spheroid cultures. A self-renewal assay can be performed after 5 to 7 days. The suspension cells can be re-plated for culturing and be designated as being a stable cell line once they reach about, for example, passage 30 or when they are homogeneous.

The invention will be further described in the following examples, which do not limit the scope of the invention described in the claims.

### EXAMPLES

### Example 1

### Producing BROV Media

A preparation of BROV medium was made as follows. First, 2 mL bovine pituitary extract (Part Code CC-4009G; Lonza, Verviers, Belgium), 0.5 mL human epithelial growth factor (2.5 μg in BROV) (Part Code CC-4017G; Lonza, Verviers, Belgium), 0.5 mL hydrocortisone (0.25 mg in BROV) (Part Code CC-4031G; Lonza, Verviers, Belgium), and 0.5 mL insulin (2 mg in BROV) (Part Code CC-4021G; Lonza, Verviers, Belgium) were added to 500 mL of MEBM Basal Media without Phenol Red (Part Code CC-3153; Lonza, Verviers, Belgium) to form a medium designated MEGM. The resulting 503.5 mL of MEGM was added to 500 mL of DMEM/F12 (50:50) medium (Catalog No. 16-405-CV; Mediatech, Inc., A Corning Subsidiary, Manassas, Va.) that contained 182.55 mg of L-glutamine and no phenol red.

This combined 1003.5 mL mixture was supplemented with HEPES buffer (10 mM; Catalog No. 25-060-CI, Mediatech, Inc.), Antibiotic-Antimycotic Solution (100 I.U. per mL penicillin; 100 μg/mL streptomycin; 0.25 μg/mL amphotericin) (Catalog No. 30-004-CI, Mediatech, Inc., A Corning Subsidiary, Manassas, Va.), 1× non-essential amino acids (Catalog No. 25-025-CI, Mediatech, Inc., A Corning Subsidiary, Manassas, Va.), 1 nM beta-estradial (Catalog No. E2758, Sigma-Aldrich, St. Louis, Mo.), 2% fetal bovine serum (Catalog No. SH30071.03, Thermo Fisher Scientific Inc., Logan, Utah), 0.1% AlbuMAX®I (Catalog No. 11020-021, Gibco® products from Life Technologies Corporation, Grand Island, N.Y.), 0.5× B-27 (Catalog No. 17504-044, Gibco® products from Life Technologies Corporation, Grand Island, N.Y.), and 0.5× N-2 (Catalog No. 17502-048, Gibco® products from Life Technologies Corporation, Grand Island, N.Y.).

The final volume was 1081.5 mL, which contained 500 mL of MEBM Basal Media, 500 mL of DMEM/F12, and the ingredients listed in Table 2 at the indicated final concentrations using the B-27 at 0.5× and the N-2 at 0.5×.

### Example 2

### Culturing Cells with BROV Medium

BROV medium was produced as described in Example 1 to be a cell culture medium for culturing breast, prostate, and ovarian primary cultures in order to create cell lines for pre-clinical models for cancer research. Most cancer cell models that are available are over 20 years old and many “younger” cell lines are cross-contaminated with well-established cell lines. This poses a problem on breast, ovarian, and prostate cancer research due to limitations on available pre-clinical models especially since creating immortalized cell lines can be tedious and time-consuming and can have a high failure rate (e.g., less than 2 percent success rate).

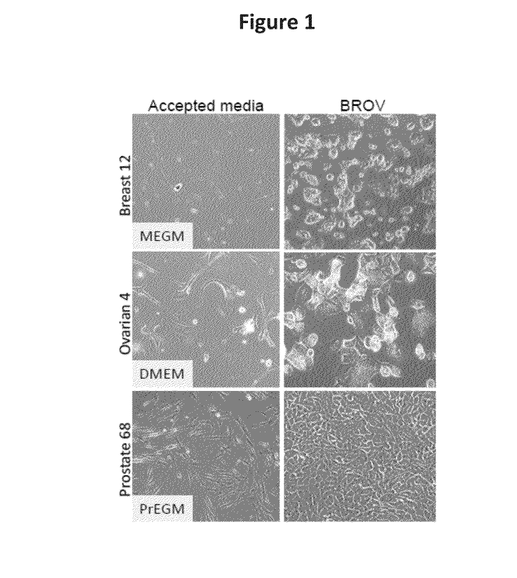

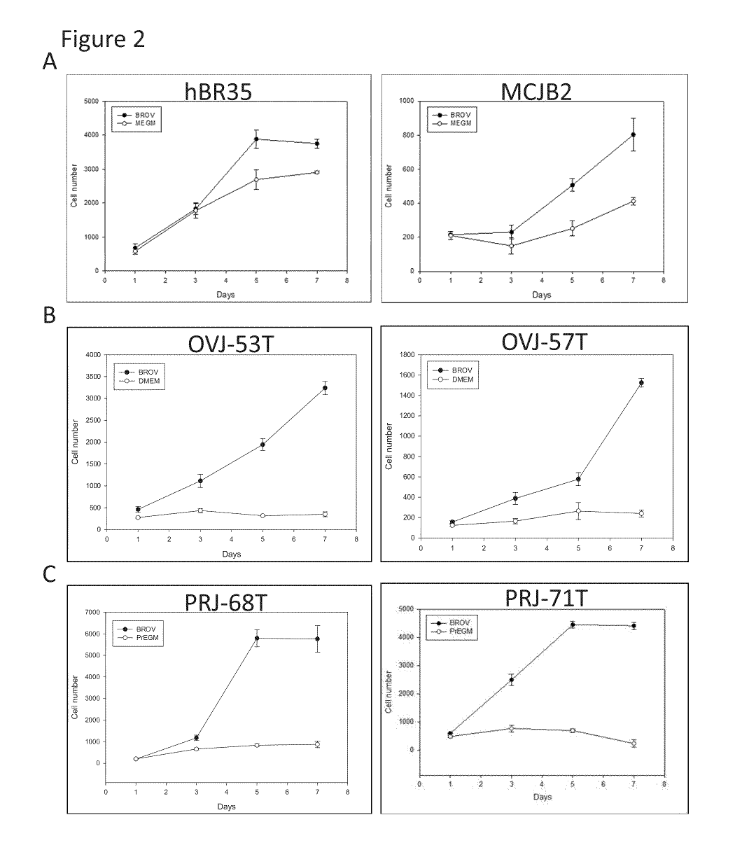

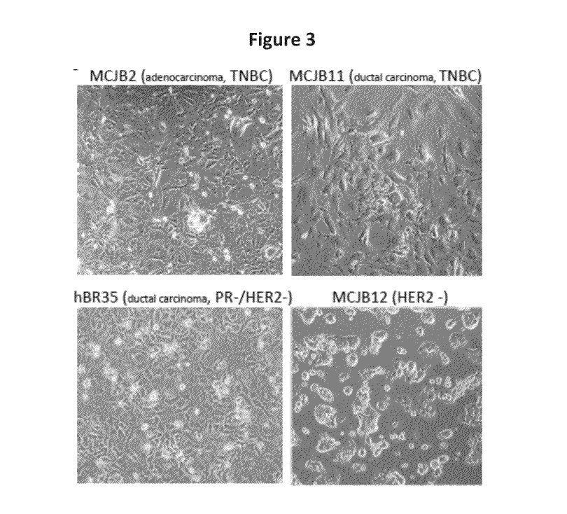

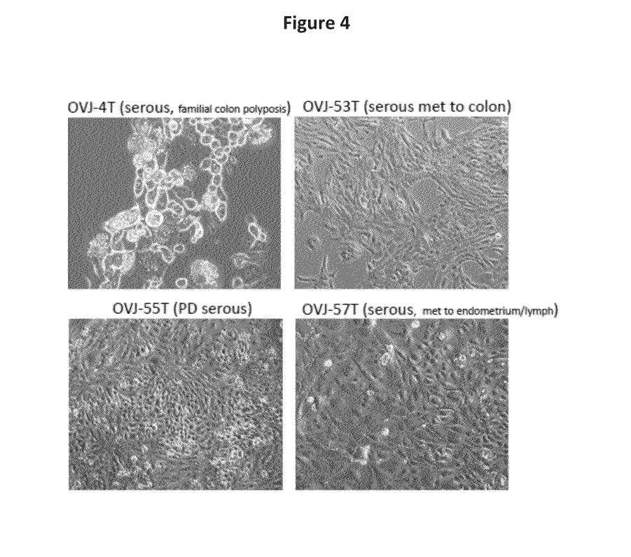

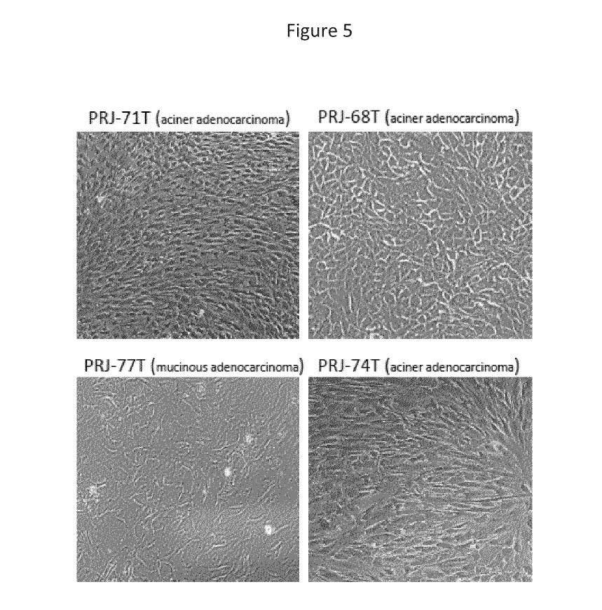

Use of the BROV medium significantly increased the success rate of creating immortalized cell lines to 5.5% for breast cells, to 7.8% for ovarian cells, and to 13.7% for prostate cells. In addition to increasing the success rate of creating immortalized cell lines, the BROV medium, in comparison to other traditional media, improved the phenotype and growth rate of the cells. BROV medium was used in parallel to MEGM for breast cells, DMEM+10% FBS for ovarian cells, and PrEGM (CC-3166, Lonza, Verviers, Belgium) for prostate cells. In all cases, the other media promoted stromal phenotypes; whereas, the BROV medium promoted epithelial phenotypes (FIG. 1). In addition, proliferative rates were significantly higher in cells grown in BROV medium a verses the other media (FIGS. 2A-2C, breast, ovarian, and prostate, respectively). The BROV medium also promoted strong adherent epithelial phenotypes in breast, ovarian, and prostate cultures (FIGS. 3-5). The BROV medium was tested using renal, lung, and bladder primary cultures. In these cases, it did not significantly affect phenotype or proliferative rates. Taken together, these results demonstrate that BROV medium can be used to create immortalized cell lines and to culture immortalized cell lines.

### Example 3

### Producing αBROV Media

A preparation of αBROV medium was made as follows. First, 2 mL bovine pituitary extract (Part Code CC-4009G; Lonza, Verviers, Belgium), 0.5 mL human epithelial growth factor (2.5 μg in αBROV) (Part Code CC-4017G; Lonza, Verviers, Belgium), 0.5 mL hydrocortisone (0.25 mg in αBROV) (Part Code CC-4031G; Lonza, Verviers, Belgium), and 0.5 mL insulin (2 mg in αBROV) (Part Code CC-4021G; Lonza, Verviers, Belgium) were added to 500 mL of αMEM (Catalog No. SH30265.01; Thermo Scientific, Logan, Utah) to form a medium designated αBROV. The resulting 503.5 mL of αBROV was added to 500 mL of DMEM/F12 (50:50) medium (Catalog No. 16-405-CV; Mediatech, Inc., A Corning Subsidiary, Manassas, Va.) that contained 182.55 mg of L-glutamine and no phenol red.

This combined 1003.5 mL mixture was supplemented with HEPES buffer (10 mM; Catalog No. 25-060-CI, Mediatech, Inc.), Antibiotic-Antimycotic Solution (100 I.U. per mL penicillin; 100 μg/mL streptomycin; 0.25 μg/mL amphotericin) (Catalog No. 30-004-CI, Mediatech, Inc., A Corning Subsidiary, Manassas, Va.), 1× non-essential amino acids (Catalog No. 25-025-CI, Mediatech, Inc., A Corning Subsidiary, Manassas, Va.), 1 nM beta-estradial (Catalog No. E2758, Sigma-Aldrich, St. Louis, Mo.), 2% fetal bovine serum (Catalog No. SH30071.03, Thermo Fisher Scientific Inc., Logan, Utah), 0.1% AlbuMAX®I (Catalog No. 11020-021, Gibco® products from Life Technologies Corporation, Grand Island, N.Y.), 0.5× B-27 (Catalog No. 17504-044, Gibco® products from Life Technologies Corporation, Grand Island, N.Y.), and 0.5× N-2 (Catalog No. 17502-048, Gibco® products from Life Technologies Corporation, Grand Island, N.Y.).

The final volume was 1081.5 mL, which contained 500 mL of αMEM, 500 mL of DMEM/F12, and the ingredients listed in Table 2 at the indicated final concentrations.

### Example 4

### Culturing Cells with αBROV Medium

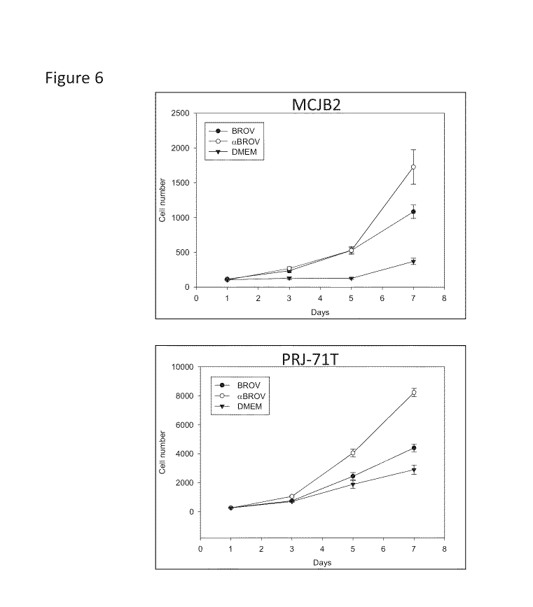

αBROV medium was produced as described in Example 3 to be a cell culture medium for culturing breast and prostate cells. In addition, the ability of αBROV to promote proliferation of breast (MCBJ2 cells) and prostate (PRJ-71T cells) cells was assessed and compared to ability of BROV medium and DMEM to promote proliferation of breast and prostate cells. The proliferative rates of MCBJ2 cells and PRJ-71T cells were significantly higher for cells grown in αBROV medium as compared to the other media (FIG. 6). Taken together, these results demonstrate that BROV and αBROV media can be used to create immortalized cell lines and can be used to culture immortalized cell lines.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A culture medium preparation for creating an immortalized breast, ovarian, or prostate cell line, wherein said preparation comprises:
(a) at least three compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine, and
(b) at least three compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

2. The preparation of claim 1, wherein said preparation comprises at least four compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine.

3. The preparation of claim 1, wherein said preparation comprises at least five compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine.

4. The preparation of claim 1, wherein said preparation comprises at least six compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine.

5. The preparation of claim 1, wherein said preparation comprises at least seven compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine.

6. The preparation of claim 1, wherein said preparation comprises at least four compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

7. The preparation of claim 1, wherein said preparation comprises at least five compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

8. The preparation of claim 1, wherein said preparation comprises at least six compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

9. The preparation of claim 1, wherein said preparation comprises at least seven compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

10. A method for creating an immortalized breast, ovarian, or prostate cell line, wherein said method comprises culturing breast, ovarian, or prostate primary cells in the presence of a culture medium preparation, wherein said preparation comprises:
(a) at least three compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine, and
(b) at least three compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

11. The method of claim 10, wherein said preparation comprises at least four compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine.

12. The method of claim 10, wherein said preparation comprises at least five compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine.

13. The method of claim 10, wherein said preparation comprises at least six compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine.

14. The method of claim 10, wherein said preparation comprises at least seven compounds selected from the group consisting of from about 5 mg/mL to about 15 mg/mL of thymidine, from about 5 mg/mL to about 15 mg/mL of 2′-deoxyadenosine, from about 5 mg/mL to about 15 mg/mL of 2′-deoxycytidine, from about 5 mg/mL to about 15 mg/mL of 2′-deoxyguanosine, from about 5 mg/mL to about 15 mg/mL of uridine, from about 5 mg/mL to about 15 mg/mL of adenosine, from about 5 mg/mL to about 15 mg/mL of cytidine, from about 5 mg/mL to about 15 mg/mL of guanosine, from about 5 mg/mL to about 15 mg/mL of α-lipoic acid, from about 5 mg/mL to about 15 mg/mL of sodium pyruvate, from about 0.5 mg/mL to about 5 mg/mL of pyridoxine, and from about 0.5 mg/mL to about 5 mg/mL of hypoxanthine.

15. The method of claim 10, wherein said preparation comprises at least four compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

16. The method of claim 10, wherein said preparation comprises at least five compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

17. The method of claim 10, wherein said preparation comprises at least six compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

18. The method of claim 10, wherein said preparation comprises at least seven compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

19. The method of claim 10, wherein said preparation comprises at least eight compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.

20. The method of claim 10, wherein said preparation comprises at least nine compounds selected from the group consisting of from about 0.005 μg/mL to about 0.05 μg/mL of catalase, from about 1 mg/mL to about 200 mg/mL of insulin, from about 0.001 μM to about 1 μM of superoxide dismutase, from about 0.01 mM to about 0.05 mM of ethanolamine, from about 1 μg/mL to about 200 μg/mL of L-carnitine, from about 0.01 mg/mL to about 1 mg/mL of linoleic acid, from about 0.01 mg/mL to about 1 mg/mL of linolenic acid, from about 1 nM to about 100 nM of progesterone, from about 1 μM to about 500 μM of putrescine, from about 1 nM to about 50 nM of sodium selenite, and from about 0.1 nM to about 10 nM of triodo-I-thyronine.
